(19) United States
(12) Patent Application Publication
(10) Pub. No.: US 2011/0172692 A1
(43) Pub. Date: Jul. 14, 2011

(54) HEMODIALYSIS ARTERIO-VENOUS GRAFT WITH A RING-LIKE DIAMETER-ADJUSTABLE DEVICE

(76) Inventor: Steven Wu, Lexington, MA (US)

(21) Appl. No.: 12/997,832

(22) PCT Filed: Jun. 15, 2009
(86) PCT No.: PCT/US09/47318

§ 371 Date: Mar. 29, 2011

Related U.S. Application Data
(60) Provisional application No. 61/061,402, filed on Jun. 13, 2008.

(51) Int. Cl. A61M 5/00

(52) U.S. Cl. 606/191

(57) ABSTRACT

A ring device is integrated externally onto and around a regular hemodialysis arterio-venous graft conduit. The ring device is adjustable to reduce or to normalize (by rebounding) the diameter of the graft, and eventually to change blood flow through the graft. This ring device is designed such that it can be scaled up or down to change its diameter, and simultaneously the diameter of the associated graft prior to and even after its implantation into a patient's arm or leg. Since a hemodialysis arterio-venous graft is usually implanted superficially underneath skin, the device and its adjustable parts can be electively or urgently located and operated from outside by touching and pushing the skin, subcutaneous tissues and the device without a surgical incision. The ring device can be located anywhere within the graft and can function with a single ring or as a set of multiple rings.

### CROSS-REFERENCE TO RELATED APPLICATIONS

This application claims priority from U.S. Patent Application No. 61/061,402 filed Jun. 13, 2008.

### STATEMENT REGARDING FEDERALLY SPONSORED RESEARCH

Not applicable.

### FIELD OF THE INVENTION

The invention relates to the field of hemodialysis devices in general and in particular to a hemodialysis arterio-venous graft with a ring device for adjusting the inside diameter of the arterio-venous graft.

### BACKGROUND OF THE INVENTION

Twenty six million Americans have chronic kidney disease. While some of these patients undergo treatment to maintain some kidney functions, some patients completely lose their kidney function, referred to as end-stage kidney disease, and rely on artificial kidney through hemodialysis, to stay alive. In the United States, there are approximately 400,000 end-stage kidney disease patients currently receiving hemodialysis. The cost for hemodialysis is about 26 billion dollars a year. Approximately 15% of the total cost is spent on hemodialysis vascular access. Vascular access is long considered to be the Achilles heel of dialysis. There are three basic kinds of vascular access for hemodialysis, arterio-venous (AV) fistula, an arterio-venous graft, and a venous catheter. Hemodialysis patients who do not have adequate veins for a fistula, become candidates for an arterio-venous graft or a venous catheter which is strongly discouraged due to its high morbidity and mortality. An arterio-venous graft is an example of this type of access. The graft is created by connecting an artery to a vein using a synthetic tube of a biocompatible material such as GORE-TEX® polytetrafluoroethylene (PTFE). The graft becomes an artificial vein that can be used repeatedly for needle placement and blood access during hemodialysis. A shortcoming of this technique is, dialysis patients with a graft frequently have thrombosed grafts partially due to poor blood flow. Another shortcoming of this technique results in dialysis patients occasionally experiencing hand ischemia due to high blood flow into the graft (arterial steal syndrome). It is technically a challenge for a surgeon to decide, and to create, the right size arterial anastomosis. An arterial anastomosis is the connection point between a graft and the side of an artery. A slight difference in the size of an arterial anastomosis could cause either a high flow into the graft, resulting in hand ischemia, or a low flow into the graft, resulting in thrombosis. A variation in the size of an arterial anastomosis may not be easily controlled surgically.

Current grafts have a fixed diameter from an arterial inlet to a venous outlet. The inlet of graft is usually sutured to the side of an artery which then supplies blood to both the graft and the extremity to which the graft is attached, either a hand or a leg. As described above, incorrectly sized arterial anastomosis can result in an imbalance of blood flow into the graft and the extremities of a dialysis patient. Too much blood flow into the graft can cause less blood into the extremity, thereby inducing hand or leg ischemia and/or tissue loss. On the other hand, too little blood into the graft can cause thrombosis of the graft. Thrombosis of the graft is a very common problem known to anyone skilled in the art, and it is partially secondary to poor arterial inflow besides the stenosis at venous anastomosis.

Considering the limitations of previous arterio-venous grafts, an improved design is needed.

### SUMMARY OF THE INVENTION

The present invention overcomes the aforementioned drawbacks by providing a means for adjusting the inside diameter of an arterio-venous graft. The issue of too much blood flow, resulting in hand or leg ischemia, or too little blood flow resulting in thrombosis of the graft, are minimized by installing the ring device of the current invention during surgical placement of an arterio-venous graft. The ring device is implanted superficially underneath the skin along with the graft. The ring device and its adjustable parts are easily located and operated from outside by touching and pushing the skin and subcutaneous tissues without surgical incision. One of the devices can be accessed with a syringe through the skin and subcutaneous tissues. With this adjustable ring device, the surgeon is allowed to create a reasonable size arterial anastomosis without worrying about hand ischemia (steal syndrome). If hand ischemia occurs, the diameter of the ring can be decreased in the operation room or after placement of an arterio-venous graft. By applying the ring(s) in different location(s) along the graft, blood flow and/or intra-graft pressure can be regulated within different segments of the graft.

The ring device of the current invention can be placed at any point along the length of the implanted graft. In one embodiment, multiple ring devices are placed at conveniently spaced distances along the length of the graft.

In one aspect, the invention provides an apparatus for interconnecting an artery and a vein. The apparatus includes a tubular graft wherein the tubular graft has an inside diameter, a first end configured for attachment to the artery, and a second end configured for interconnection with the vein such that the artery and vein can be placed in fluid communication. The apparatus further includes a ring device suitable for adjusting the inside diameter of the tubular graft. The tubular graft can be constructed to receive needles configured for transporting blood for hemodialysis. The tubular graft can be placed superficially underneath the skin of a patient, and adjustment of the ring device can be achieved from outside the skin layer.

In one form, the ring device includes an annular member having one or more outer interlocking teeth on a distal end of the annular member, and one or more inner interlocking teeth on a proximal end of the annular member. At least one outer interlocking tooth and at least one inner interlocking tooth are configured such that the one outer interlocking tooth and the one inner interlocking tooth engage to form a closed loop. The distal end can include a plurality of interlocking teeth dimensioned to engage a plurality of interlocking teeth on the proximal end to form the closed loop.

In another form, the ring device includes an annular member having a tooth on a distal end of the annular member and a hole in a proximal end of the annular member such that the tooth can engage the hole to form a closed loop. A plurality of holes can be provided on the proximal end of the annular member such that the tooth can engage different holes to form a closed loop of varying inside diameters.

In yet another form, a longitudinal cross-section of an inner wall of the ring device has generally a polygonal shape for engaging the tubular graft and for adjusting the inside diameter of the tubular graft when the inside diameter of the ring device is adjusted. The ring device can include a wall having an inner surface with a rib that extends away from the inner surface of the wall. A longitudinal cross-section of the rib can have a generally polygonal shape. The rib can be centered about a lateral axis of the wall. A longitudinal cross-section of the rib can have a triangular shape. The rib can have an edge furthest from the inner surface of the wall wherein the edge is located adjacent or on a lateral axis of the wall. The rib can have an edge furthest from the inner surface of the wall wherein the edge is located spaced from a lateral axis of the wall.

In still another form, the ring device includes an elastic tubular body and a valve configured to engage a pumping device for varying fluid pressure inside the tubular body such that the inside diameter of the tubular body can be varied by varying fluid pressure inside the tubular body. The valve can include a self-sealing septum suitable for engaging a hollow needle of a syringe such that fluid can be injected into or aspirated from the interior of the tubular body to vary the inside diameter of the tubular body. The ring device can be supplied with the syringe. The valve can include a valve body in fluid communication with the interior of the tubular body, and a ball valve in the valve body, and the ring device can be provided with a flexible bag for introducing a fluid though the valve and into the tubular body to vary the inside diameter of the tubular body. The valve can be a flapper valve covering a fluid port in fluid communication with the interior of the tubular body. The ring device can be provided with a flexible bag for introducing a fluid past the valve and into the tubular body.

In another aspect, the invention provides a method for interconnecting an artery and a vein. The method uses a tubular graft having an arterial end and a venous end. In the method, a ring device is affixed around the graft. An opening is created in the vein, and the venous end of the graft is attached to the opening in the vein. An opening is created in the artery, and the arterial end of the graft is attached to the opening in the artery. This creates an alternate passage for the flow of blood from the artery through the tubular graft to the vein. In the method, the ring device can be configured for adjusting an inside diameter of the graft. The tubular graft can be constructed to receive needles configured for transporting blood for hemodialysis. The tubular graft can be placed superficially underneath the skin of a patient, and adjustment of the ring device can be achieved from outside the skin layer.

In the method, the ring device can include an annular member having one or more outer interlocking teeth on a distal end of the annular member, and one or more inner interlocking teeth on a proximal end of the annular member. At least one outer interlocking tooth and at least one inner interlocking tooth are configured such that the one outer interlocking tooth and the one inner interlocking tooth engage to form a closed loop. The distal end can include a plurality of interlocking teeth dimensioned to engage a plurality of interlocking teeth on the proximal end to form the closed loop.

In another version of the method, the ring device includes an annular member having a tooth on a distal end of the annular member and a hole in a proximal end of the annular member such that the tooth can engage the hole to form a closed loop. A plurality of holes can be provided on the proximal end of the annular member such that the tooth can engage different holes to form a closed loop of varying inside diameters.

In yet another version of the method, a longitudinal cross-section of an inner wall of the ring device has generally a polygonal shape for engaging the tubular graft and for adjusting the inside diameter of the tubular graft when the inside diameter of the ring device is adjusted. The ring device can include a wall having an inner surface with a rib that extends away from the inner surface of the wall. A longitudinal cross-section of the rib can have a generally polygonal shape. The rib can be centered about a lateral axis of the wall. A longitudinal cross-section of the rib can have a triangular shape. The rib can have an edge furthest from the inner surface of the wall wherein the edge is located adjacent or on a lateral axis of the wall. The rib can have an edge furthest from the inner surface of the wall wherein the edge is located spaced from a lateral axis of the wall.

In still another version of the method, the ring device includes an elastic tubular body and a valve configured to engage a pumping device for varying fluid pressure inside the tubular body such that the inside diameter of the tubular body can be varied by varying fluid pressure inside the tubular body. The valve can include a self-sealing septum suitable for engaging a hollow needle of a syringe such that fluid can be injected into or aspirated from the interior of the tubular body to vary the inside diameter of the tubular body. The ring device can be supplied with the syringe. The valve can include a valve body in fluid communication with the interior of the tubular body, and a ball valve in the valve body, and the ring device can be provided with a flexible bag for introducing a fluid though the valve and into the tubular body to vary the inside diameter of the tubular body. The valve can be a flapper valve covering a fluid port in fluid communication with the interior of the tubular body. The ring device can be provided with a flexible bag for introducing a fluid past the valve and into the tubular body.

It is therefore an advantage of the invention to provide an apparatus and method for interconnecting an artery and a vein wherein means for adjusting the inside diameter of an arterio-venous graft is provided. The means for adjusting the inside diameter of the graft can be an adjustable ring device that is dimensioned to be placed around the graft such that the ring device can be adjusted to engage a wall of the graft and adjust the inside diameter of the arterio-venous graft. With this adjustable ring device, the surgeon is allowed to create a reasonable size arterial anastomosis without worrying about hand ischemia. If hand ischemia occurs, the diameter of the ring device can be decreased in the operating room, or after placement of the arterio-venous graft. Thus, blood flow and/or intra-graft pressure can be regulated at any time after placement of the graft with the surrounding ring device. The adjustability of the ring device accommodates the physiological characteristics of the patient. There is no need for an open surgical procedure to adjust the inside diameter of the graft.

The foregoing and other advantages of the invention will appear in the detailed description that follows. In the description, reference is made to the accompanying drawings that illustrate exemplary embodiments of the invention.

### BRIEF DESCRIPTION OF THE DRAWINGS

FIG. 1 is a depiction of a right brachial artery to right antecubital vein arterio-venous graft.

FIG. 2 is a depiction of a hemodialysis arterio-venous graft and hemodialysis circuit.

FIG. 3A is a depiction of the ring device of the current invention in an open configuration.

FIG. 3B is a depiction of the ring device of FIG. 3A in a closed configuration.

FIG. 4A is a side view of the ring device of FIG. 3A installed on a tubular graft in a default mode.

FIG. 4B is a side view of the ring device of FIG. 3A installed on a tubular graft in a rebound mode (normalizing diameter).

FIG. 4C is a side view of the ring device of FIG. 3A installed on a tubular graft in a contraction mode (decreased diameter).

FIG. 5 is a depiction of a hemodialysis arterio-venous graft with a single external ring device; and

FIG. 6 is an illustration of a arterio-venous graft with a multiple of the ring device of the current invention.

FIG. 7A shows a longitudinal cross-sectional view of a second embodiment of a ring device of the current invention installed on a graft.

FIG. 7B shows a longitudinal cross-sectional view of a third embodiment of a ring device of the current invention installed on a graft.

FIG. 7C shows a longitudinal cross-sectional view of a fourth embodiment of a ring device of the current invention installed on a graft.

FIG. 7D shows a longitudinal cross-sectional view of a fifth embodiment of a ring device of the current invention installed on a graft.

FIG. 7E shows a longitudinal cross-sectional view of a sixth embodiment of a ring device of the current invention installed on a graft.

FIG. 8A is a perspective view of a seventh embodiment of a ring device of the current invention installed on a tubular graft in a default mode.

FIG. 8B is a detailed partial perspective view of the ring device of FIG. 8A before movement to a first contraction mode.

FIG. 8C is a detailed partial perspective view of the ring device of FIG. 8A during movement to the first contraction mode.

FIG. 8D is a detailed partial perspective view of the ring device of FIG. 8A in a first contraction mode.

FIG. 8E is a detailed partial perspective view of the ring device of FIG. 8A before movement to a second contraction mode.

FIG. 8F is a detailed partial perspective view of the ring device of FIG. 8A during movement to the second contraction mode.

FIG. 8G is a detailed partial perspective view of the ring device of FIG. 8A in a second contraction mode.

FIG. 8H is a side view of the ring device of FIG. 8A in the default mode.

FIG. 8I is a side view of the ring device of FIG. 8A in the first contraction mode.

FIG. 8J is a side view of the ring device of FIG. 8A in the second contraction mode.

FIG. 9A is a perspective view of an eighth embodiment of a ring device of the current invention installed on a tubular graft.

FIG. 9B is a side view of the ring device of FIG. 9A in the default mode.

FIG. 9C is a side view of the ring device of FIG. 9A in the contraction mode.

FIG. 9D is a partial side view of the ring device of FIG. 9A wherein fluid is being injected into the ring device of FIG. 9A in order to decrease the inside diameter of the ring device of FIG. 9A.

FIG. 9E is a partial side view of the ring device of FIG. 9A wherein fluid is being aspirated from the ring device of FIG. 9A in order to increase the inside diameter of the ring device of FIG. 9A.

FIG. 10A is a perspective view of a ninth embodiment of a ring device of the current invention installed on a tubular graft.

FIG. 10B is a side view of the ring device of FIG. 10A in the default mode.

FIG. 10C is a side view of the ring device of FIG. 10A in the contraction mode.

FIG. 10D is a partial cross-sectional side view of the valve of the ring device of FIG. 10A.

FIG. 10E is a partial cross-sectional side view of the valve of the ring device of FIG. 10A during release of fluid from the ring device.

FIG. 10F is a partial cross-sectional side view of the valve of a tenth embodiment of a ring device of the current invention.

FIG. 10G is a partial cross-sectional side view of the valve of the ring device of FIG. 10F during release of fluid from the ring device.

Like reference numerals will be used to refer to like parts from Figure to Figure in the following description of the drawings.

### DETAILED DESCRIPTION OF THE INVENTION

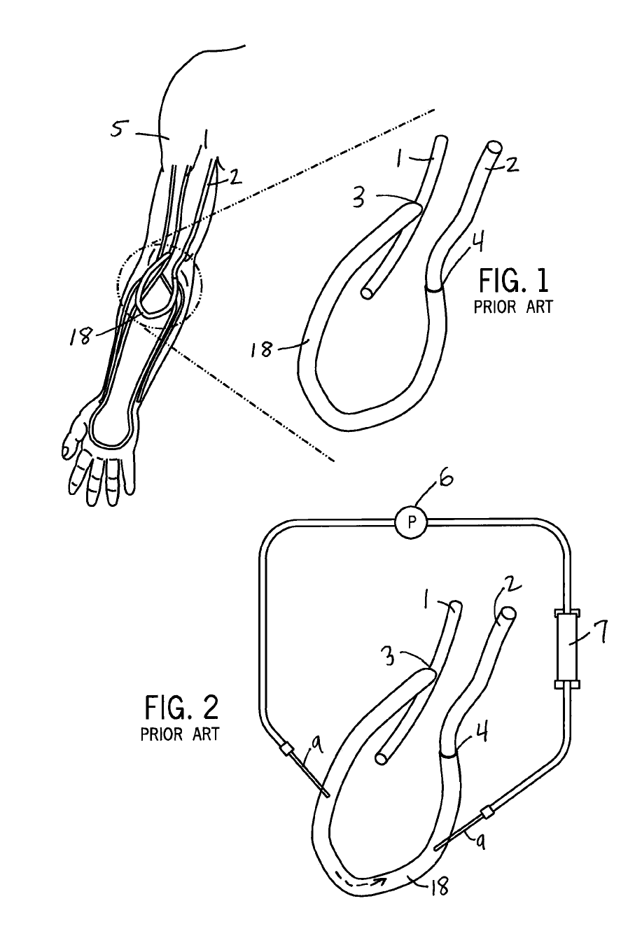

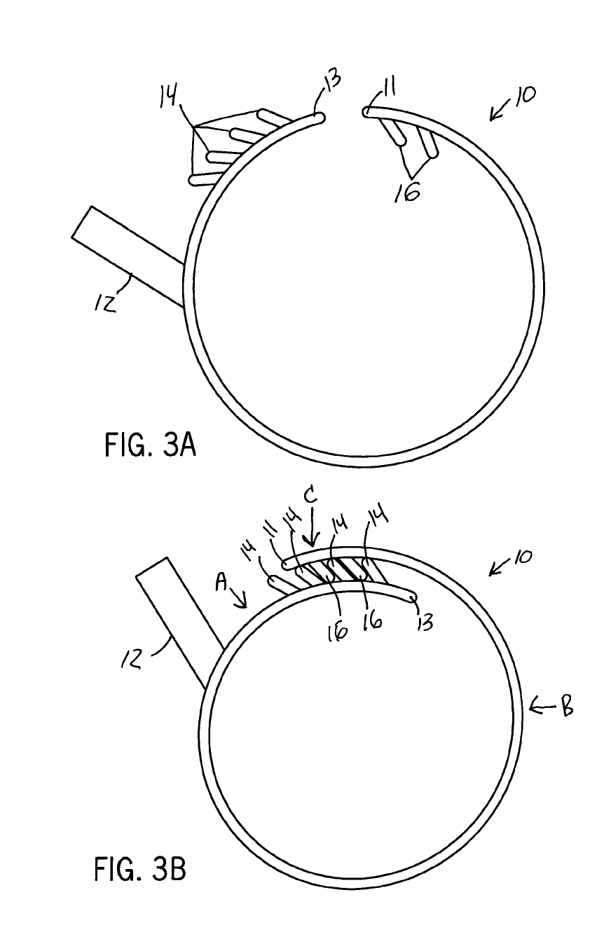

Referring to FIG. 1, a depiction of a right brachial artery 1 to a right antecubital vein 2 arterio-venous graft 18 is displayed. The arterio-venous graft 18 is shown with an arterial anastomosis 3 at the artery 1, and a venous anastomosis 4 at the vein 2 in a right arm 5. By implementing the arterio-venous graft 18 in a hemodialysis patient as shown in FIG. 2, an alternate path for blood flow is created. The graft 18 functions as an artificial vessel into which needles 9 can be repeatedly inserted to allow blood to be removed from a patient's blood stream. The blood is then returned to the patient's blood stream after it has passed through a hemodialysis machine. The arterio-venous graft 18 is usually implanted superficially underneath a patient's skin. In FIG. 2, a diagram of a hemodialysis arterio-venous tubular graft 18 and a hemodialysis circuit is shown. Hemodialysis generally involves the passage of blood from an artery through a dialysis machine, illustrated by a pump 6 and an artificial kidney 7 in FIG. 2. The dialysis machine cleans the blood of impurities. As described above, it is important to be able to control the blood flow through an implanted tubular graft 18.

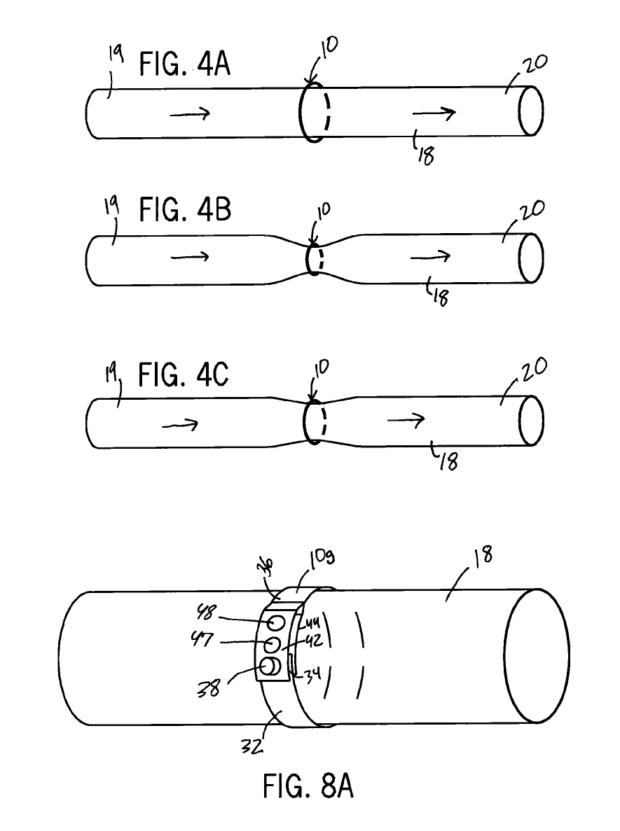

Referring to FIG. 3A, a line drawing representation of a ring device 10 of the current invention is shown. The ring device 10 is configured to operate in various operational modes including, open mode, contract mode, and rebound mode. As discussed, the ability to control the blood flow rate in hemodialysis treatment is critical to the health of the dialysis patient as well as the patency of the graft itself. The current invention provides a means to easily and effectively control the blood flow rate during dialysis treatment. Prior to hemodialysis treatment using an arterio-venous graft, a vascular access is usually prepared to allow for easier and more efficient removal and replacement of a patient's blood. During this preparation at least one of the ring device 10 is integrated externally onto and around a tubular arterio-venous graft 18 (see FIG. 1). Ring device 10 is generally installed in its default mode, wherein the proximal end 11 and the distal end 13 are disengaged as shown in FIG. 3A or engaged as a closed loop or circle but in default mode. While a patient is undergoing hemodialysis treatment, the ring device 10 is used to adjust the inside diameter of the tubular arterio-venous graft 18. The ring device 10 is adjustable to reduce or to normalize (by rebounding) the inside diameter of the tubular arterio-venous graft 18. This adjustment results in varying the blood flow through the arterio-venous graft 18. The ring device 10 can be formed from a biocompatible, elastic polymeric or metallic material.

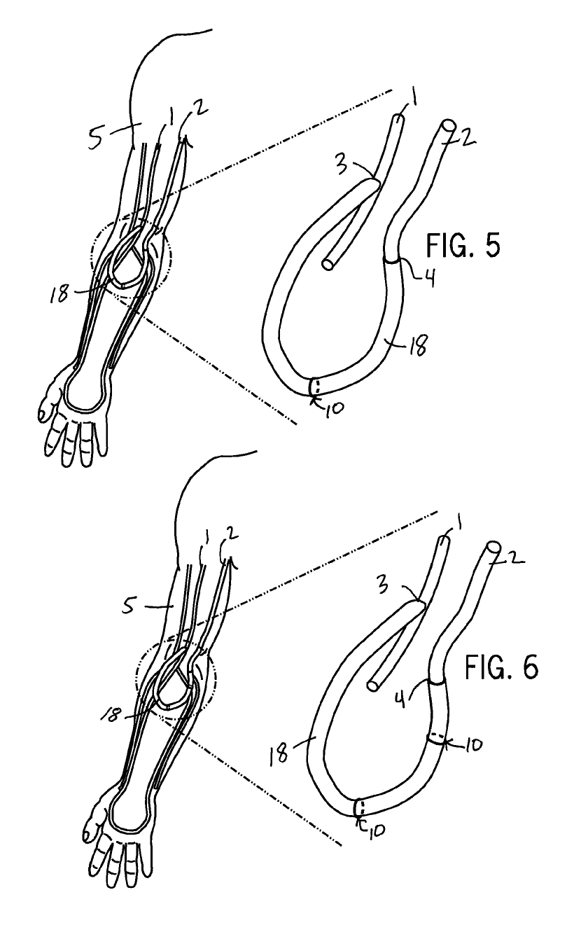

The ring device 10 is configured to be scaled up, in rebound mode, as shown in FIG. 4C thereby normalizing the inside diameter of the arterio-venous graft 18. In another exemplary application, the ring device is scaled down in contract mode, as shown in FIG. 4B, thereby decreasing the inside diameter of the tubular arterio-venous graft 18. Also incorporated in ring device 10 is at least one outer interlocking tooth 14 on a distal end 13 as well as at least one inner interlocking tooth 16 on a proximal end 11. By engaging the at least one outer interlocking tooth 16 on the proximal end 11 with the at least one inner interlocking tooth 14 on the distal end 13, a closed loop is formed as shown in FIG. 3B. To establish different contraction levels of a tubular arterio-venous graft 18 using the ring device 10, a plurality of outer interlocking teeth 16 and a plurality of inner interlocking teeth 14 are integrated thereon or further integrated with different interlocking teeth if the default mode is in loop or circle status. It is not necessary to form corresponding pairs of teeth between the proximal end 11 the distal of ring device 10, therefore it is not required to have the same number of teeth on each end of the ring device 10.

To facilitate the adjustability feature of the ring device 10, an outwardly extending tab 12 can be integrated proximate the distal end 13 of the ring device 10 as shown in FIG. 3A. In the embodiment wherein a plurality of outer interlocking teeth 14 and a plurality of inner interlocking teeth 16 are formed on ring device 10, the inside diameter of the tubular arterio-venous graft 18 is adjustable by engaging different sets of the at least one outer interlocking tooth 14 with at least one of the inner interlocking tooth 16. Contraction of the inside diameter of the tubular arterio-venous graft 18, is attained by a user holding the distal end 13 of the ring device 10 with a first finger and the outwardly extending tab 12 with a second finger then pushing the outwardly extending tab 12 inwards, thereby disengaging the pair of interlocking teeth, and reengaging with a different pair of interlocking teeth. Adjustment of the ring device 10 to a default state is done by reversing the steps for contraction. This adjustment is achieved by a user holding the proximal end 11 of the ring device with a first finger and the outwardly extending tab 12 of the ring device with a second finger and pushing the outwardly extending tab 12 inwardly and then outward. As the arterio-venous tubular graft 18 and the ring device 10 are implanted underneath the skin, adjustment of the inside radius of the tubular graft 18 is from outside by operably touching and pushing the skin, subcutaneous tissues and the ring device 10, without a surgical incision. The ring device 10 is constructed in a manner such that it can be easily identified and operated underneath a layer of clothing. The ring device 10 can be advantageously located at any point along the length of the tubular arterio-venous graft 18, particularly near the arterio-graft anastomosis.

Referring to FIGS. 4A-4C, a depiction of a number of modes of the ring device 10 is shown. FIG. 4A depicts a tubular graft 18 with the ring device of the current invention mounted thereon. The arrows illustrate the direction of blood flow through the tubular arterio-venous graft 18, from an arterial end 19 to a venous end 20. The ring device 10, in FIG. 4A depicts the default mode of ring device 10. Referring to FIG. 4B, the ring device 10 is shown in a contract mode, thereby decreasing the inside diameter of the tubular graft 18 as discussed with reference to FIG. 3B. FIG. 4C depicts the ring device 10 in rebound mode. In the rebound mode, ring device 10 is not actively involved in adjusting the diameter of the tubular arterio-venous graft 18. The movement from default to contraction mode is also shown in FIG. 3B. While pushing C inward using one finger, compressing both A and B simultaneously inward (or together) will change interlocking, positions of both ends, teeth, and this will cause the ring to contract. The movement from contraction mode to default mode is as follows. Without pushing C, further compressing both A and B inward (or together) will allow interlocking teeth to dislocate to its default position.

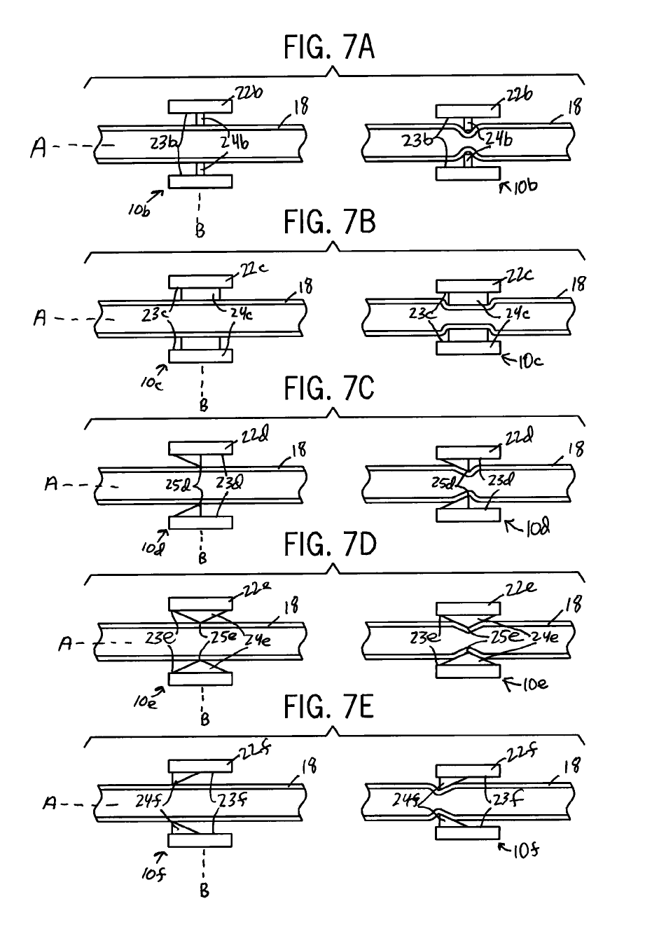

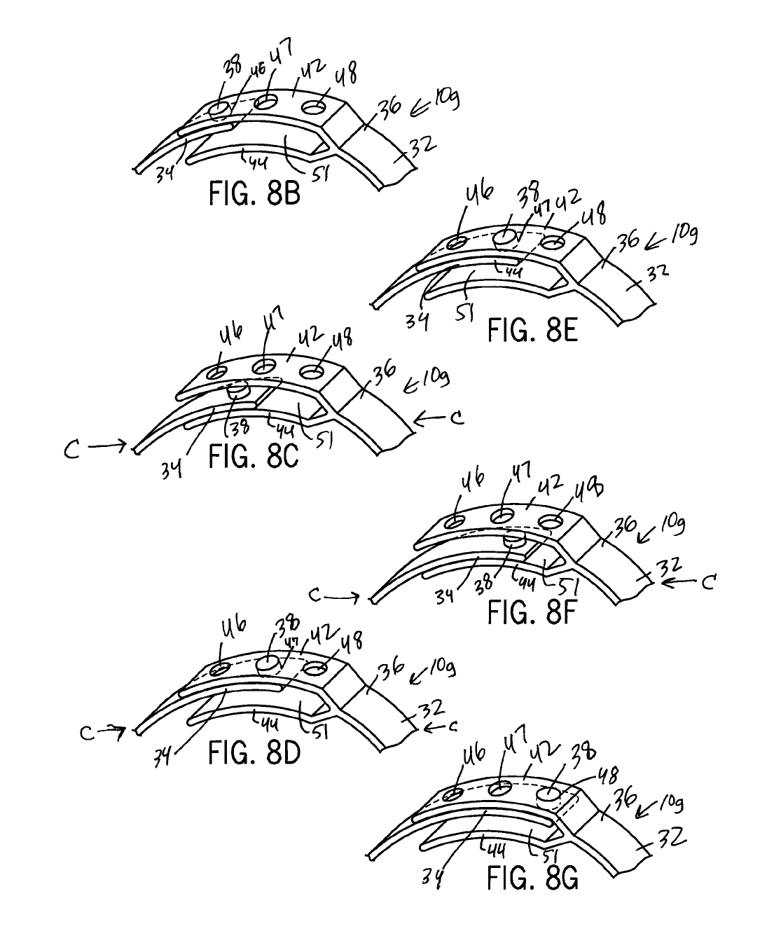

In one embodiment of the current invention, FIG. 5 illustrates a diagram of a right brachial artery 1 to a right antecubital vein 2 arterio-venous graft 18 with the ring device 10. The ring device 10 can be typically located near the inlet end of the graft (near the arterio-graft anastomosis) to regulate blood flow for management of arterial steal syndrome. Another embodiment of the current invention is shown in FIG. 6, illustrating the use of multiple ring devices 10 mounted along the length of the tubular arterio-venous graft 18. The ring device 10 can be mounted anywhere along the length of the tubular graft 10. The number of ring devices needed in any implementation is only limited by the application in which it is being used. By applying the ring(s) to different locations along the graft 18, blood flow and intra-graft pressure can be regulated within different segments of the graft, which is divided by the ring(s). Similarly, the ring device 10 functions as effectively whether the tubular graft is placed in an arm or a leg of a patient.

The new ring device 10 serves as a size (or diameter)-guarding device used to control blood flow through the tubular arterio-venous graft 18 regardless the size of the arterial anastomosis. Advantageously, with this new ring device 10, a surgeon can liberally create a large size arterial anastomosis without worrying about hand ischemia in a patient. In the event that hand ischemia is not an issue for some patients, the ring device 10 also allows for the adjustment of optimal blood flow throughout the tubular graft 18 based on hemodialysis parameters and medical conditions. As discussed, unregulated and unnecessary high blood flow through the tubular arterio-venous graft 18 can cause other medical problems such as high-output heart failure.

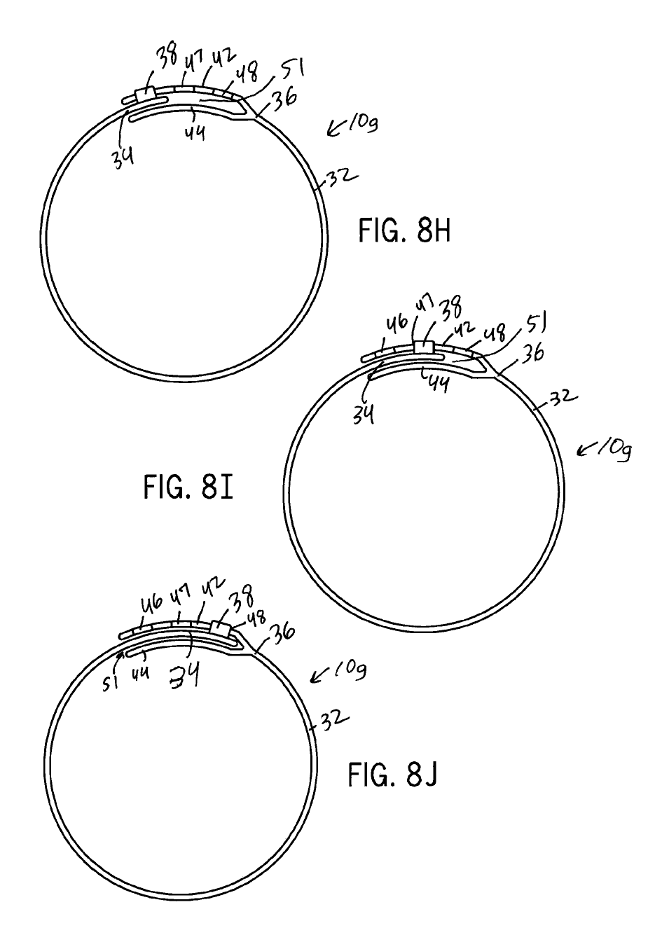

In alternative embodiments, the ring device can adjust both the diameter and the configuration of the graft conduit. The configuration will influence the dynamics of flow through the graft conduit. Specifically, looking at FIGS. 7A-7E, there are longitudinal cross-sectional views in which second to sixth embodiments of the ring device according to the invention are shown in graft 18 having a longitudinal axis A. The ring devices 10b to 10f are shown in FIGS. 7A-7E with a default mode on the left hand side and a contraction mode on the right hand side wherein the inside diameter of the tubular graft 18 is decreased as explained above.

In the configuration of FIG. 7A, the ring device 10b includes a wall 22b having a generally rectangular longitudinal cross-section. The wall 22b has an inner surface 23b and further includes a rib 24b that extends away from the inner surface 23b of the wall 22b. The rib 24b can extend circumferentially from the interlocking teeth on a proximal end 11 of the ring device 10b to a distal end 13 of the ring device 10b. The circumferential rib 24b is located centered about a lateral axis B of the wall 22b. A longitudinal cross-section of the rib 24b has a generally rectangular shape.

In the configuration of FIG. 7B, the ring device 10c includes a wall 22c having a generally rectangular longitudinal cross-section. The wall 22c has an inner surface 23c and further includes a rib 24c that extends away from the inner surface 23c of the wall 22c. The rib 24c can extend circumferentially from the interlocking teeth on a proximal end 11 of the ring device 10c to a distal end 13 of the ring device 10c. The circumferential rib 24c is located centered about a lateral axis B of the wall 22c. A longitudinal cross-section of the rib 24c has a generally rectangular shape, albeit of a different shape than rib 24b.

In the configuration of FIG. 7C, the ring device 10d includes a wall 22d having a generally rectangular longitudinal cross-section. The wall 22d has an inner surface 23d and further includes a rib 24d that extends away from the inner surface 23d of the wall 22d. The rib 24d can extend circumferentially from the interlocking teeth on a proximal end 11 of the ring device 10d to a distal end 13 of the ring device 10d. A longitudinal cross-section of the rib 24d has a generally triangular shape. The rib 24d has an edge 25d furthest from the inner surface 23d of the wall 22d, and the edge 25d is located adjacent or on a lateral axis B of the wall 22d.

In the configuration of FIG. 7D, the ring device 10e includes a wall 22e having a generally rectangular longitudinal cross-section. The wall 22e has an inner surface 23e and further includes a rib 24e that extends away from the inner surface 23e of the wall 22e. The rib 24e can extend circumferentially from the interlocking teeth on a proximal end 11 of the ring device 10e to a distal end 13 of the ring device 10e. A longitudinal cross-section of the rib 24e has a generally triangular shape. The rib 24e has an edge 25e furthest from the inner surface 23e of the wall 22e, and the edge 25e is located adjacent or on a lateral axis B of the wall 22e.

In the configuration of FIG. 7E, the ring device 10f includes a wall 22f having a generally rectangular longitudinal cross-section. The wall 22f has an inner surface 23f and further includes a rib 24f that extends away from the inner surface 23f of the wall 22f. The rib 24f can extend circumferentially from the interlocking teeth on a proximal end 11 of the ring device 10f to a distal end 13 of the ring device 10f. A longitudinal cross-section of the rib 24f has a generally triangular shape. The rib 24f has an edge 25f furthest from the inner surface 23f of the wall 22f, and the edge 25f is located longitudinally spaced from a lateral axis B of the wall 22f. The ring devices (10b through 10f, FIG. 7) can be stabilized with soft, easy-compressible material(s) filling around the rib (24b through 24f) in contraction mode, the filling compressible material(s) will be compressed and the rib will cut into the graft to regulate the diameter and/or configuration of the graft.

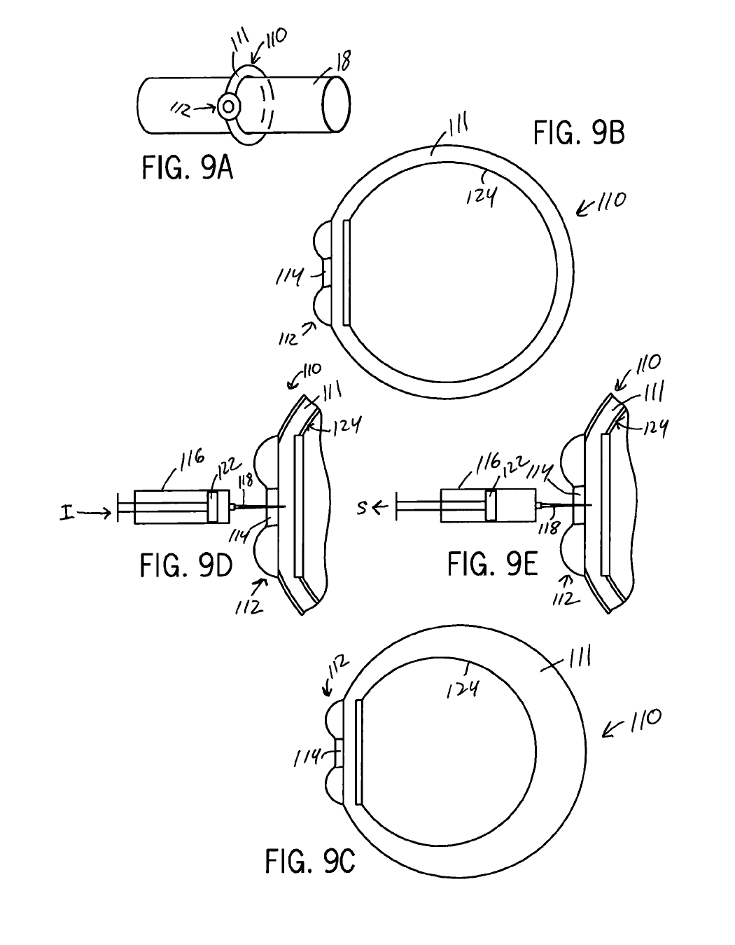

Referring now to FIGS. 8A to 8J, there is shown another embodiment of a ring device 10g according to the invention. The ring device 10g serves as a size (or diameter)-guarding device used to control blood flow through the tubular arterio-venous graft 18 regardless the size of the arterial anastomosis. The ring device 10g includes a circular body 32 having a distal end 34 and a proximal end 36. The distal end 34 has a tooth 38 that extends away from an outer surface of the distal end 34. The proximal end 36 has an upper extension plate 42 spaced from a lower extension plate 44. The upper extension plate 42 has three in-line holes 46, 47, 48.

When the ring device 10g is in the default mode of FIGS. 8A and 8H, the tooth 38 is arranged in the hole 46. In the first contraction mode of FIG. 8I, the pressure of the interior surface of the ring device 10g on the graft 18 thereby decreases the inside diameter of the tubular graft 18. In the second contraction mode of FIG. 8J, the pressure of the interior surface of the ring device 10g on the graft 18 further decreases the inside diameter of the tubular graft 18.

Movement of the ring device 10g from the default mode of FIGS. 8A and 8H to the first contraction mode of FIG. 8I to the second contraction mode of FIG. 8J is as follows. In FIG. 8B, the tooth 38 is pushed down from hole 46 into the sheath 51 between the upper extension plate 42 and the lower extension plate 44. In FIGS. 8C and 8D, compression in inward directions C causes the tooth 38 to slide toward hole 47. In FIG. 8E, the tooth 38 will slide into hole 47 thereby placing the ring device 10g in the first contraction mode shown in FIG. 8I. In FIG. 8F, the tooth 38 is pushed down from hole 47 into the sheath 51 between the upper extension plate 42 and the lower extension plate 44, and compression in inward directions C causes the tooth 38 to slide toward hole 48. In FIG. 8G, the tooth 38 will slide into hole 48 thereby placing the ring device 10g in the second contraction mode of FIG. 8J.

In FIG. 8G, the tooth 38 can be pushed down from hole 48 into the sheath 51 between the upper extension plate 42 and the lower extension plate 44, and recoil force can cause the tooth 38 to slide toward hole 47. In FIG. 8E, the tooth 38 will slide into hole 47 thereby placing the ring device 10g back in the first contraction mode of FIG. 8I. In FIG. 8D, the tooth 38 can be pushed down from hole 47 into the sheath 51 between the upper extension plate 42 and the lower extension plate 44, and recoil force can cause the tooth 38 to slide toward hole 46. In FIG. 8B, the tooth 38 will slide into hole 46 thereby placing the ring device 10g back in the default mode of FIG. 8H. The number of the holes (such as 46, 47, 48) may vary such that more or less than two contraction modes can be provided.

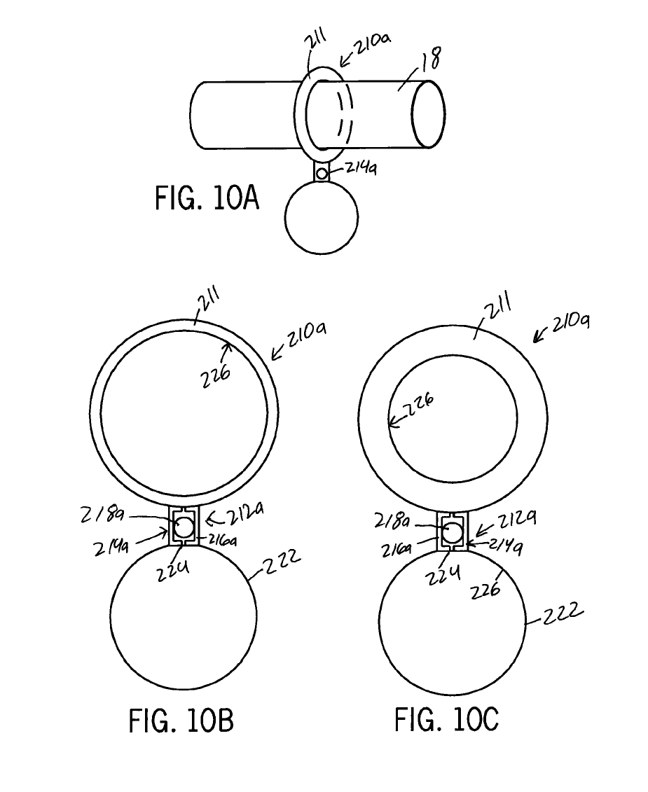

Turning now to FIGS. 9A to 9E, there is shown another ring device 110 according to the invention. The ring device 110 serves as a size (or diameter)-guarding device used to control blood flow through the tubular arterio-venous graft 18 regardless the size of the arterial anastomosis. The ring device 110 includes a toroidal balloon tube 111 that is formed from an elastic material such that the balloon tube 111 can be repeatedly inflated with a fluid and then deflated. The balloon tube 111 can be inflated evenly (inwardly and outwardly) or it can be only or more inflatable inwardly due to the elasticity of inner and outer layers of the balloon tube 111. The ring device 110 further includes a fluid access port 112 having a self-sealing septum 114. The port 112 and septum 114 form a valve that controls the introduction and release of a fluid from the tube 111. A piston pump syringe 116 having a hollow needle 118 is also provided for use with the ring device 110.

FIG. 9A depicts a tubular graft 18 with the ring device 110 mounted thereon in the default mode. FIG. 9B depicts the default mode of ring device 110. Referring to FIG. 9C, the ring device 110 is shown in a contraction mode which would decrease the inside diameter of the tubular graft 18 as discussed above with reference to FIG. 4B. FIG. 9D shows how the ring device can be transformed from the default mode of FIG. 9B to the contraction mode of FIG. 9C. A user inserts the needle 118 of the syringe 116 through the self-sealing septum 114 and into the interior of the tube 111. Saline or air or another suitable fluid is injected through the needle 118 into the tube 111 by pressing the plunger 122 of the syringe 116 toward the needle 118 in direction I. The tube 111 inflates from the injected fluid thereby decreasing the diameter of the interior surface 124 of the tube 111. The pressure of the interior surface 124 of the tube 111 on the graft 18 thereby decreases the inside diameter of the tubular graft 18. In FIG. 9E, it is shown that the tube 111 can be deflated to the default condition of FIG. 9B by inserting the needle 118 of the syringe 116 through the self-sealing septum 114 and into the interior of the tube 111 and then aspirating the fluid from the interior of the tube 111 by retracting the plunger 122 of the syringe 116 away the needle 118 in direction S. Thus, the ring device 110 can serve as a size (or diameter)-guarding device used to control blood flow through the tubular arterio-venous graft 18. The port 112 can be also a separate part connected to the circled balloon tube 111 with a connecting tube. The port 112 can be implanted in a different location from the balloon tube ring based on convenience and/or medical conditions or needs.

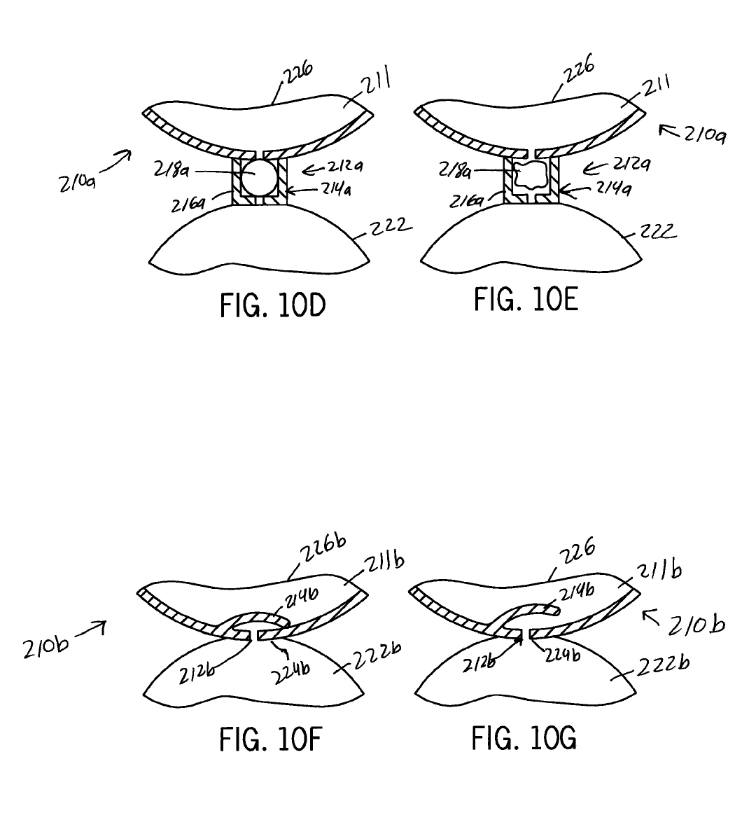

Turning now to FIGS. 10A-10E, there is shown another ring device 210a according to the invention. The ring device 210a serves as a size (or diameter)-guarding device used to control blood flow through the tubular arterio-venous graft 18 regardless the size of the arterial anastomosis. The ring device 210a includes a toroidal balloon tube 211 that is formed from an elastic material such that the balloon tube 211 can be repeatedly inflated with a fluid and then deflated. FIG. 10A depicts a tubular graft 18 with the ring device 210a mounted thereon in the default mode. FIG. 10B depicts the default mode of the ring device 210a. Referring to FIG. 10C, the ring device 210a is shown in a contraction mode which would decrease the inside diameter of the tubular graft 18 as discussed above with reference to FIG. 4B.

The ring device 210a further includes a fluid access port 212a having a valve 214a including a valve body 216a having a deformable ball valve 218a. The valve 214a controls the introduction and release of a fluid from the tube 211. A fluid bag 222 having mouth 224 for sealingly engaging the fluid access port 212a is also provided for use with the ring device 210a. FIG. 10D shows how the ring device 210a can be transformed from the default mode of FIG. 10B to the contraction mode of FIG. 10C. A user engages the mouth 224 of the fluid bag 222 with the fluid access port 212a. Saline or air or another suitable fluid is squeezed from the bag 222 through the valve body 216a passed a deforming ball valve 218a and into the tube 211. The tube 211 inflates from the injected fluid thereby decreasing the inside diameter of the interior surface 226 of the tube 211. The pressure of the interior surface 226 of the tube 211 on the graft 18 thereby decreases the inside diameter of the tubular graft 18. In FIG. 10E, it is shown that the tube 211 can be deflated to the default condition of FIG. 10B by deforming the ball valve 218a (by pressing on the valve body 216a) allowing the fluid from the interior of the tube 211 to reenter the bag 222. Thus, the ring device 210a can serve as a size (or diameter)-guarding device used to control blood flow through the tubular arterio-venous graft 18.

Looking now at FIGS. 10F and 10G, another ring device 210b according to the invention includes a fluid access port 212b having a flapper valve 214b. The flapper valve 214b controls the introduction and release of a fluid from the tube 211b forming a ring device as in FIG. 10A. The fluid bag 222b having mouth 224b can sealingly engage around a fluid access port 212b. FIG. 10F shows how the ring device 210b can be transformed from a default mode as in FIG. 10B to a contraction mode as in FIG. 100. A user engages the mouth 224b of the fluid bag 222b around the fluid access port 212b. Saline or air or another suitable fluid is squeezed from the bag 222b through the fluid access port 212b and past the opened flapper valve 214b into the tube 211b. The tube 211b inflates from the injected fluid thereby decreasing the diameter of the interior surface 226b of the tube 211b. The pressure of the interior surface 226b of the tube 211b on the graft 18 thereby decreases the inside diameter of the tubular graft 18. In FIG. 10G, it is shown that the tube 211b can be deflated to the default condition as in FIG. 10B by deforming the flapper valve 214b by pressing allowing the fluid from the interior of the tube 211b to reenter the bag 222b. Thus, the ring device 210b can serve as a size (or diameter)-guarding device used to control blood flow through the tubular arterio-venous graft 18.

The ring(s) can be integrated with the graft at the time of its implantation. Or, it can be integrated with the graft by fixing onto the graft as a piece. The ring(s) may have a sealing cover sheath, which fully wraps the rings(s), and prevents the ring(s) from being interfered with by surrounding subcutaneous tissues. The sheath allows the ring(s) to function fully and also prevents body fluid and/or blood from contracting the ring(s).

Although the ring is used for the graft, the ring(s) can also be used for hemodialysis arterio-venous fistula, which is the connection between the side of an artery and the end of a vein. By implanting the ring(s) onto and around the vein, it can regulate blood flow into the vein as well.

It is specifically intended that the present invention not be limited to the embodiments and illustrations contained herein, but include modified forms of those embodiments including portions of the embodiments and combinations of elements of different embodiments as fall within the scope of the following claims.

### INDUSTRIAL APPLICABILITY

The present invention provides a ring device for adjusting the inside diameter of a hemodialysis arterio-venous graft.

What is claimed is:

1. An apparatus for placing an artery and a vein in fluid communication, the apparatus comprising:
a tubular graft, the tubular graft having an inside diameter and having a first opening for attachment to the artery for placing the tubular graft in fluid communication with the artery and having a second opening for attachment to the vein for placing the tubular graft in fluid communication with the vein; and
a ring device dimensioned to surround and engage the tubular graft, the ring device being adjustable between a first configuration defining a first inside diameter for the ring device and a second configuration defining a second inside diameter for the ring device such that the inside diameter of the tubular graft has a different size depending on whether the ring device is in the first configuration or the second configuration.

2. The apparatus of claim 1 wherein:
the ring device includes an annular body having a proximal end and a distal end, the distal end including a first tooth, the proximal end including a second tooth, and the first tooth and the second tooth being dimensioned to engage each other to form a closed loop.

3. The apparatus of claim 1 wherein:
the ring device includes an annular body having a proximal end and a distal end, the distal end including a tooth, the proximal end including a plurality of holes such that the tooth can engage different holes to form a closed loop of varying inside diameters.

4. The apparatus of claim 1 wherein:
the ring device includes an elastic tubular body and the ring device further includes a valve configured to engage a pumping device for varying fluid pressure inside the elastic tubular body.

5. The apparatus of claim 4 wherein:
the valve is selected from a self-sealing septum suitable for engaging a hollow needle of a syringe, a ball valve, and a flapper valve covering a fluid port.

6. An apparatus for varying an inside diameter of a tubular graft in fluid communication with an artery and a vein, the apparatus comprising:
a ring device dimensioned to surround and engage the tubular graft, the ring device being adjustable between a first configuration defining a first inside diameter for the ring device and a second configuration defining a second inside diameter for the ring device such that the inside diameter of the tubular graft has a different size depending on whether the ring device is in the first configuration or the second configuration.

7. The apparatus of claim 6 wherein:
the ring device includes an annular body having a proximal end and a distal end, the distal end including a first tooth, the proximal end including a second tooth, and the first tooth and the second tooth being dimensioned to engage each other to form a closed loop.

8. The apparatus of claim 6 wherein:
the ring device includes an annular body having a proximal end and a distal end, the distal end including a tooth, the proximal end including a plurality of holes such that the tooth can engage different holes to form a closed loop of varying inside diameters.

9. The apparatus of claim 6 wherein:
the ring device includes an elastic tubular body and the ring device further includes a valve configured to engage a pumping device for varying fluid pressure inside the elastic tubular body.

10. The apparatus of claim 9 wherein:
the valve is selected from a self-sealing septum suitable for engaging a hollow needle of a syringe, a ball valve, and a flapper valve covering a fluid port.
